(19) United States
(12) Patent Application Publication
(10) Pub. No.: US 2024/0189493 A1
(43) Pub. Date: Jun. 13, 2024

(54) DEVICES FOR DETECTING INFECTION FROM PERITONEAL DIALYSIS

(71) Applicant: AWAK Technologies Pte Ltd, Singapore (SG)

(72) Inventors: Suresha BELUR VENKATARAYA, Singapore (SG); Mandar Manohar GORI, Singapore (SG); Jason Tze Chern LIM, Singapore (SG); Yue WANG, Singapore (SG); Peter HAYWOOD, Singapore (SG); Ricardo AGUILAR GRAJEDA, Singapore (SG); Alessandro DONADIO, Singapore (SG)

(73) Assignee: AWAK Technologies Pte Ltd, Singapore (SG)

(21) Appl. No.: 18/286,456

(22) PCT Filed: Apr. 14, 2022
(86) PCT No.: PCT/SG2022/050222

§ 371 Date: Oct. 11, 2023

(30) Foreign Application Priority Data
Apr. 15, 2021 (SG) 10202103858V

(51) Int. Cl. A61M 1/16

(52) U.S. Cl. CPC A61M 1/1605; A61M 2205/331

(57) ABSTRACT

The present disclosure generally relates to a device (200) for detecting infection in a patient (102) undergoing peritoneal dialysis. The device (200) comprises: a housing module (202) removably coupleable to a fluidic element (204) configured for receiving waste dialysate fluid (130) from the patient (102); a set of lighting elements (206) disposed on the housing module (202) and configured for emitting light into the fluidic element (204); a set of optical sensors (208) disposed on the housing module (202) and configured for measuring optical properties of the light that has interacted with the waste dialysate fluid (130) in the fluidic element (204); and a control module configured for measuring turbidity of the waste dialysate fluid (130) based on the optical properties, wherein the dialysate turbidity is indicative of infection in the patient (102) if the dialysate turbidity and historical dialysate turbidity of the patient (102) satisfy a set of predefined conditions.

### CROSS REFERENCE TO RELATED APPLICATION(S)

The present disclosure claims the benefit of Singapore Patent Application No. 10202083858V filed on 15 Apr. 2021, which is incorporated in its entirety by reference herein.

### TECHNICAL FIELD

The present disclosure generally relates to devices for detecting infection from peritoneal dialysis. More particularly, the present disclosure describes various embodiments of devices for detecting infection such as peritonitis in a patient undergoing peritoneal dialysis.

### BACKGROUND

Millions of people worldwide suffer from kidney-related problems, such as chronic kidney disease (CKD) and end-stage renal disease (ESRD), and they require either dialysis or transplantation to maintain life. There are two modalities of dialysis—haemodialysis and peritoneal dialysis. In haemodialysis, blood is pumped out of the patient's body to a dialysis machine which filters the blood and returns the filtered blood to the body. In peritoneal dialysis, the peritoneum in the patient's abdomen acts as a natural filtration membrane. While dialysis provides a way of survival in case of kidney failure, it is still associated with a major change in quality of life. Performing dialysis at home rather than in a clinical setting can help improve patient's quality of life as this can afford some normalization of daily routines as patients can plan their dialysis around their activities. However, there are challenges with home-based dialysis. The patient or the patient's caregiver needs to learn how to perform home-based dialysis on their own, and especially for peritoneal dialysis, the operational steps can be quite cumbersome.

FIG. 1 illustrates an exemplary peritoneal dialysis apparatus 100 used by a patient 102 at home. The patient 102 has a transfer set 104 including a catheter that is inserted into the patient's abdomen. To begin peritoneal dialysis, the patient 102 connects the transfer set 102 to a common tubing or line or patient line 106 that leads to a tubing connector 108. The transfer set 104 has a valve 105 to open and close the catheter which should normally be closed to prevent infection. A fresh bag 110 containing fresh dialysis fluid or solution is connected to the tubing connector 108 via a supply tubing or fill line 112. The fill line 112 has a valve 114 for opening and closing the fill line 112 at appropriate stages during peritoneal dialysis. A drain bag 116 is connected to the tubing connector 108 via a drain tubing or drain line 118. The drain line 118 has a valve 120 for opening and closing the drain line 118 at appropriate stages during peritoneal dialysis.

During peritoneal dialysis, the fresh dialysis fluid flows from the fresh bag 110 into the abdomen where the peritoneum allows waste compounds and excess fluid to pass from the blood into the fresh dialysis fluid. The fresh dialysis fluid contains a sugar such as glucose/dextrose that acts as the main osmotic agent to achieve fluid removal or filtration across the peritoneum into the abdominal cavity. The used dialysis fluid is later discharged from the body as waste dialysate which contains the waste compounds and excess fluid. The waste dialysate is collected in the drain bag 116 and thrown away. The apparatus 100 may be configured to perform the fluid exchange by gravity. Alternatively, the apparatus 100 may include a machine or pump 122 to perform the fluid exchange, such as when the patient 102 is sleeping.

The apparatus 100 allows the patient 102 to receive peritoneal dialysis treatment at home or on the go without significantly compromising his/her quality of life. However, the patient 102 should be careful when handling the apparatus 100 to avoid contamination. For example, the patient 102 may not properly connect the transfer set 104 to the common line 106, such as due to insufficient training, and risk hand touch contamination on the tips of the transfer set 104 and/or common line 106. Such contamination could result in infection in the patient 102, such as peritonitis. Peritonitis can be detected through visual observation of the cloudiness or turbidity of the waste dialysate in the drain bag 116, as well as observing for any symptoms related to abdominal pain. However, observing the cloudiness of the waste dialysate fluid is very subjective, and minor changes in the cloudiness may not be easily noticed by the naked eye. Different patients 102 may have different vision acuity for this task as well. Early-stage peritonitis can result in mild cloudiness that go unnoticed by the patient 102, resulting in delayed diagnosis and treatment. Hence, there is inconsistency in the quality of checking across patients 102, with some peritonitis cases being missed out and some non-peritonitis cases being false positives. Moreover, some patients 102 with peritonitis may not show symptoms during the early stages, and symptoms like mild abdominal pain can be falsely attributed to other factors, leading to delayed in diagnosis and treatment. It could just take a few days for the cloudiness to become more obvious and/or symptoms to become more serious. But this also means peritonitis has also evolved to a more advanced stage, making treatment more difficult which can result in increased mortality rate.

Therefore, in order to address or alleviate at least one of the aforementioned problems and/or disadvantages, there is a need to provide improved devices for detecting infection from peritoneal dialysis.

### SUMMARY

According to a first aspect of the present disclosure, there is a device for detecting infection in a patient undergoing peritoneal dialysis. The device comprises:
a housing module removably coupleable to a fluidic element configured for receiving waste dialysate fluid from the patient;
a set of lighting elements disposed on the housing module and configured for emitting light into the fluidic element;
a set of optical sensors disposed on the housing module and configured for measuring optical properties of the light that has interacted with the waste dialysate fluid in the fluidic element; and
a control module configured for measuring turbidity of the waste dialysate fluid based on the optical properties,
wherein the dialysate turbidity is indicative of infection in the patient if the dialysate turbidity and historical dialysate turbidity of the patient satisfy a set of predefined conditions.

According to a second aspect of the present disclosure, there is a device for detecting infection in a patient undergoing peritoneal dialysis. The device comprises:
a housing module removably coupleable to a fluidic element configured for receiving waste dialysate fluid from the patient;
a colour sensor configured for measuring colour information of a reagent test element disposed in the fluidic element that has reacted with the waste dialysate fluid in the fluidic element; and
a control module configured for detecting infection based on the colour information, the colour information representing enzyme activity in the waste dialysate fluid that is indicative of infection in the patient.

According to a third aspect of the present disclosure, there is a device for detecting infection in a patient undergoing peritoneal dialysis. The device comprises:
a housing module removably coupleable to a fluidic element configured for receiving waste dialysate fluid from the patient;
a set of lighting elements disposed on the housing module and configured for emitting light into the fluidic element;
a set of optical sensors disposed on the housing module and configured for:
   measuring optical properties of the light that has interacted with the waste dialysate fluid in the fluidic element; and
   measuring colour information of a reagent test element disposed in the fluidic element that has reacted with the waste dialysate fluid in the fluidic element; and
a control module configured for:
   measuring turbidity of the waste dialysate fluid based on the optical properties, the dialysate turbidity indicative of infection in the patient if the dialysate turbidity and historical dialysate turbidity of the patient satisfy a set of predefined conditions; and
   detecting the infection based on the colour information, the colour information representing enzyme activity in the waste dialysate fluid that is indicative of the infection.

According to a fourth aspect of the present disclosure, there is a computerized method for detecting infection in a patient undergoing peritoneal dialysis. The method comprises:
measuring colour information of a reagent test element that has reacted with waste dialysate fluid from the patient;
extracting RGB colour data from the colour information; and
detecting infection based on the RGB colour data, the RGB colour data representing enzyme activity in the waste dialysate fluid that is indicative of infection in the patient.

Devices for detecting infection from peritoneal dialysis according to the present disclosure are thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1 is an illustration of a peritoneal dialysis apparatus.

FIGS. 2A and 2B are illustrations of a device for detecting infection based on turbidity of waste dialysate.

FIGS. 3A and 3B are illustrations of the device of FIGS. 2A and 2B in use with the peritoneal dialysis apparatus.

FIGS. 4A to 4C are illustrations of calibration of the device of FIGS. 2A and 2B.

FIG. 5 is an illustration of a device for detecting infection based on colour information of a reagent test.

FIGS. 6A and 6B are illustrations of the device of FIG. 5 in use with the peritoneal dialysis apparatus.

FIG. 7 is an illustration related to enzyme activity in waste dialysate.

FIGS. 8A and 8B are illustrations of the devices of FIGS. 2A, 2B, and 5 in use with the peritoneal dialysis apparatus.

FIG. 9 is an illustration of a device for detecting infection based on turbidity of waste dialysate and colour information of a reagent test.

FIGS. 10A and 10B are illustrations of the device of FIG. 9 in use with the peritoneal dialysis apparatus.

FIGS. 11A and 11B are other illustrations of a device for detecting infection based on turbidity of waste dialysate and colour information of a reagent test.

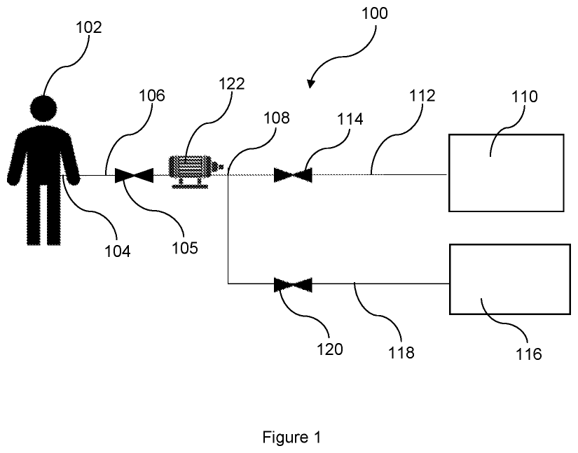

### DETAILED DESCRIPTION

For purposes of brevity and clarity, descriptions of embodiments of the present disclosure are directed to devices for detecting infection from peritoneal dialysis, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognised by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organisation of elements that mathematically exhibits a cardinality of at least one (e.g. a set as defined herein can correspond to a unit, singlet, or single-element set, or a multiple-element set), in accordance with known mathematical definitions. The terms “first”, “second”, etc. are used merely as labels or identifiers and are not intended to impose numerical requirements on their associated terms.

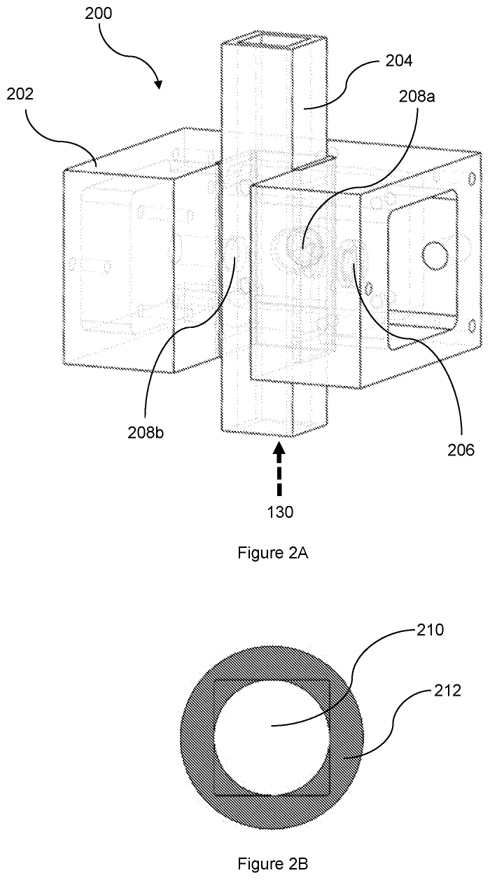

In some representative or exemplary embodiments of the present disclosure, with reference to FIGS. 2A and 2B, there is a device 200 for use with the peritoneal dialysis apparatus 100 for detecting infection in a patient 102 undergoing peritoneal dialysis. The device 200 includes a housing module 202 removably coupleable to a fluidic element 204 configured for receiving waste dialysate fluid 130 from the patient 102. The fluidic element 204 may have various cross-sectional shapes such as cylindrical. The device 200 includes a set of lighting elements 206 disposed on the housing module 202 and configured for emitting light into the fluidic element 204. The device 200 includes a set of optical sensors 208 disposed on the housing module 202 and configured for measuring optical properties of the light that has interacted with the waste dialysate fluid 130 in the fluidic element 204. The device 200 includes a control module configured for measuring turbidity of the waste dialysate fluid 130 based on the optical properties, wherein the dialysate turbidity is indicative of infection, such as peritonitis, in the patient 102 if the dialysate turbidity and historical dialysate turbidity of the patient 102 satisfy a set of predefined conditions.

In some embodiments, the lighting elements 206 include one or more light emitting diodes (LEDs). For example, the LEDs may include white light LEDs. Alternatively, the LEDs may emit light of a specific wavelength or within a narrow range of wavelengths, such as 860 nm for infrared light.

In some embodiments, the optical sensors 208 include a scattered light sensor 208a for measuring the light that has been scattered by the waste dialysate fluid 130. In some embodiments, the optical sensors 208 include a transmitted light sensor 208b for measuring the light that has been transmitted through the waste dialysate fluid 130. Notably, the transmitted light sensor 208b and lighting elements 206 are disposed on the housing module 202 such that the fluidic element 204 resides between them. In some embodiments as shown in FIG. 2A, the optical sensors 208 include both the scattered light sensor 208a and transmitted light sensor 208b. For example, the scattered light sensor 208a and transmitted light sensor 208b are located at different areas of the housing module 202 for measuring the scattered light and transmitted light, respectively. The types of optical sensors 208 may be selected based on the type of lighting elements 206. For example, the scattered light sensor 208a and transmitted light sensor 208b may use the same type of optical sensor or photodetector. The optical properties may include a relationship between the scattered light and transmitted light.

When the patient 102 is suffering from peritonitis, bacteria, mycobacteria, fungi, and parasites in the peritoneum can trigger generation of white blood cells or leukocytes which is an inflammatory marker of infection. Leukocytes typically range from 12 to 14 microns in size and accumulation of leukocytes in the waste dialysate fluid 130 would lead to increased cloudiness or turbidity of the waste dialysate fluid 130. As turbidity increases, more leucocytes in the waste dialysate fluid 130 would scatter the light emitted by the lighting elements 206 into the waste dialysate fluid 130. The scattered light sensor 208a would receive a stronger light signal while the transmitted light sensor 208b would receive a weaker light signal. Each of the light signals detected by the scattered light sensor 208a or transmitted light sensor 208b can be used individually to correlate with turbidity of the waste dialysate fluid 130. Alternatively, both light signals can be used together to derive an optical ratio which can be correlated with the turbidity. Combining light signals from the scattered light sensor 208a and transmitted light sensor 208b can improve sensitivity in the light measurements to better correlate with turbidity and consequently detect infection, as compared to individual light signals. Combining the light signals also helps to improve reliability especially if the lighting elements 206 deteriorate over time, such as decrease in brightness.

In some embodiments, the device 200 further includes a set of lenses 210 for collimating the light emitting from the lighting elements 206 into the fluidic element 204 and/or for focusing the light on the optical sensors 208. The lenses 210 help to improve the signal-to-noise ratio and sensitivity of the light measurements. For example as shown in FIG. 2B, the lenses 210, which may include one or more condenser lenses, may be disposed in front of the lighting elements 206 for collimating the light emitted therefrom into parallel beams, which would help to minimize scattering of light before the light reaches the fluidic element 204. Similarly, the lenses 210 such as condenser lenses may be disposed in front of the scattered light sensor 208a and/or transmitted light sensor 208b to focus the light onto the respective optical sensors 208. Notably, the optical sensors 208 are positioned at the focal length of the lenses 210 so that the light can be effectively focused. The lenses 210 may be disposed on a supporting base 212 that is attached to the housing module 202, such as by using an optical adhesive cured under ultraviolet light.

The fluidic element 204 is a disposable component which is replaced with every use of the apparatus 100. The device 200 is reusable for the next disposable fluidic element 204 for the next peritoneal dialysis treatment. The fluidic element 204 may be pre-sterilized using a suitable method, such as gamma rays, ethylene oxide, or electron beam, before using it for peritoneal dialysis treatment. The fluidic element 204 may be pre-installed as part of the disposable tubings used in the apparatus 100. The patient 102 needs to couple the device 200 to the fluidic element 204 before beginning peritoneal dialysis treatment.

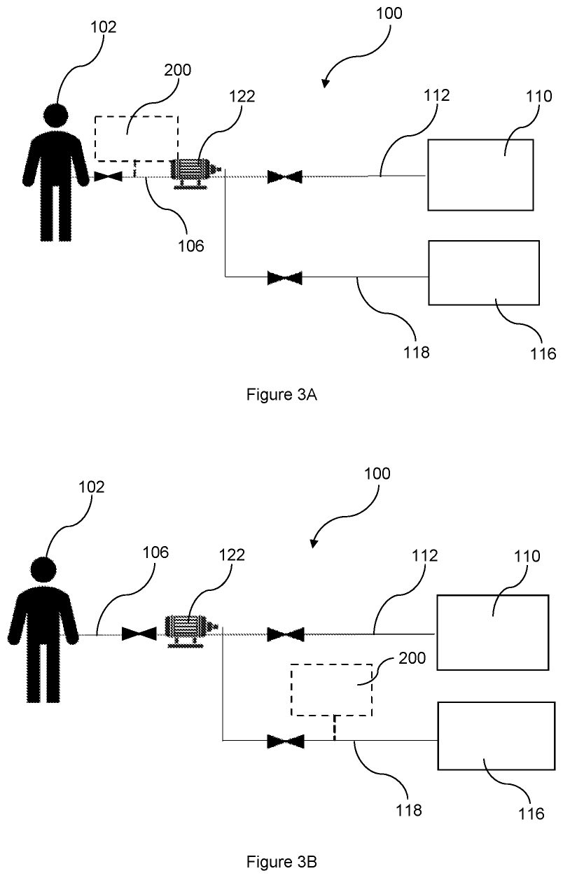

In one embodiment as shown in FIG. 3A, the fluidic element 204 is part of or connected to the common line 106 and the patient 102 couples the device 200 to the common line 106. In one embodiment as shown in FIG. 3B, the fluidic element 204 is part of or connected to the drain line 118 and the patient 102 couples the device 200 to the drain line 118.

The patient 102 can use the device 200 to measure turbidity of the waste dialysate fluid 130 discharged away from the patient 102 along the common line 106 and drain line 118 to the drain bag 116. In one example, the patient 102 can use the device 200 to measure the turbidity during the initial drain phase before starting the peritoneal dialysis treatment. The initial drain phase removes the waste dialysate fluid 130 from the previous dwell session. In another example, the patient 102 can use the device 200 to measure the turbidity during the peritoneal dialysis treatment as the waste dialysate fluid 130 flows along the common line 106 and drain line 118 to the drain bag 116. In another example, the patient 102 can use the device 200 to measure the turbidity during the final drain phase at the end of the peritoneal dialysis treatment.

The final drain phase removes the last waste dialysate fluid 130 from the abdomen before the patient 102 fills the abdomen with the fresh dialysate fluid from the fresh bag 110.

Further, the device 200 is able to measure the turbidity in the waste dialysate fluid 130 in a dynamic state as it flows through the fluidic element 204, for example at a flow rate of 100 ml/min. As such, the measurement of turbidity can be integrated with the peritoneal dialysis treatment workflow which would be less cumbersome for the patient 102 and reduce interruption in the treatment, as opposed to having to manually collect a sample of the waste dialysate fluid 130 for measurement.

The device 200 may be calibrated before the patient 102 can use it to measure turbidity and detect infection. It has been empirically found that the optical ratio (y) between the scattered light signal and transmitted light signal is linearly correlated with the turbidity values (x), in the linear equation y=mx+c. Although the sensitivity of the device 200 can be represented by the gradient (m), the light intensity of the lighting elements 206, sensitivity of the optical sensors 208, and the baseline clarity of the transparent housing module 202 may vary slightly, such that the baseline optical ratio in absence of a turbid fluid may not be the same across different devices 200.

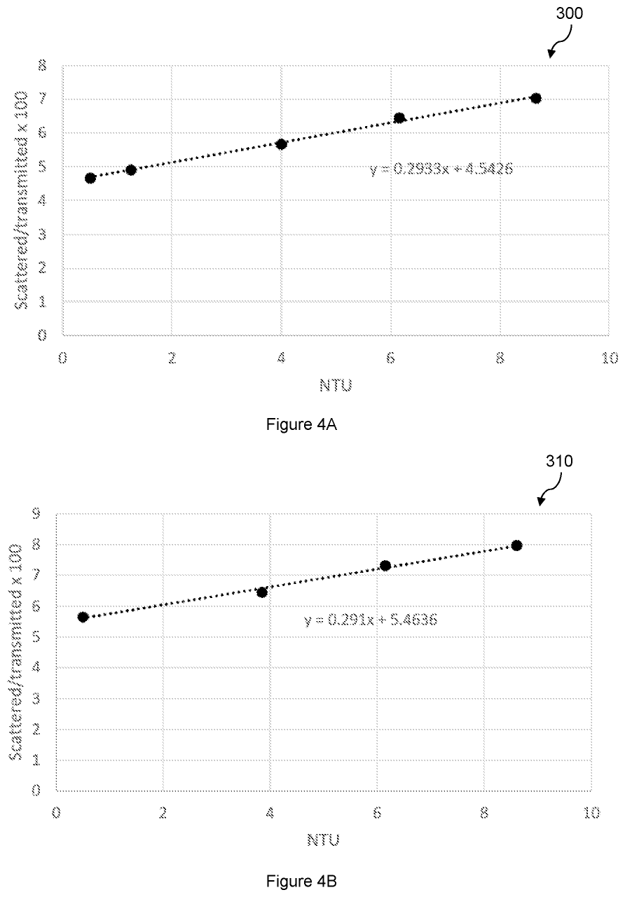

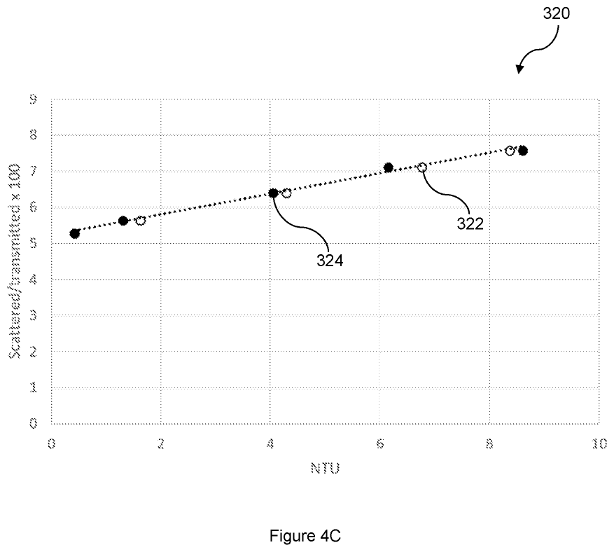

Two exemplary devices 200 with the same design but different components, such as different housing module 202, different lighting elements 206, different optical sensors 208, and different lenses 210 were used to determine the gradient (m) representing the sensitivity of the device 200. Priming fluids with known turbidity values (measured in nephelometric turbidity units (NTUs)) were used, such as Baxter Dianeal® peritoneal dialysis solutions. The priming fluids were communicated through the fluidic element 204 and the device 200 measured the optical ratio for the respective turbidity values of the priming fluids. The optical ratios (y-axis) for the respective peritoneal dialysis solutions and their respective NTUs (x-axis) are plotted in graphs 300,310 as shown in FIGS. 4A and 4B for the two devices 200. It was found that the gradient (m) is 0.2933 and 0.291, respectively, and the offset value (c) is 4.5426 and 5.4636, respectively. The results showed that both devices 200, having the same design but with different components, share the same sensitivity or slope of response based on the gradient (m) of approximately 0.29. The sensitivity is thus specific to the design of the device 200, even though different components may be used.

Since the results corroborated that devices 200 with the same design have approximately the same gradient (m), a calibration process may be performed to calibrate the device 200 before it can be used, including determining the offset value (c) for the device 200. The calibration process is done using the linear equation y=0.29x+c and a single priming fluid with known NTU, such as the fresh dialysate fluid in the fresh bag 110.

The control module, which includes a computer processor, may be configured to perform a series of computerized steps of the calibration process. The steps include measuring optical properties of the light that has interacted with the fresh dialysate fluid in the fluidic element 204, correlating the optical properties with the turbidity values of the fresh dialysate fluid, and deriving an optical-turbidity profile of the device 200 based on the correlation. The optical-turbidity profile is represented by the linear equation y=0.29x+c where the offset value (c) is now known. Once calibrated, the device 200 can be used for peritoneal dialysis and the optical-turbidity profile is configured to determine the turbidity of the waste dialysate fluid 130 from the patient 102 based on optical properties of the light that has interacted with the waste dialysate fluid 130 in the fluidic element 204.

The optical-turbidity profile of the device 200 can be used to predict the turbidity values of the waste dialysate fluid 130. As shown in the graph 320 in FIG. 4C, the predicted turbidity values 322 are plotted against measured turbidity values 324 that were measured using a commercial turbidity meter. The commercial turbidity meter is a bulky and expensive equipment that measures fluid turbidity in a static state, as opposed to the device 200 which can measure fluid turbidity in a dynamic state, i.e. when the fluid is flowing. As shown in the graph 320, it was found that the predicted turbidity values 322 and measured turbidity values 324 were very close to each other. This provides that the calibration process can effectively calibrate the devices 200 to derive the optical-turbidity profile for determining the turbidity of waste dialysate fluid 130.

As described above, the control module is configured for measuring the turbidity of the waste dialysate fluid 130 based on the optical properties such as the optical ratio. The dialysate turbidity is indicative of peritonitis if the dialysate turbidity and historical dialysate turbidity of the patient 102 satisfy a set of predefined conditions. In one example, the predefined conditions may include the dialysate turbidity being higher than the historical dialysate turbidity averaged over a predefined duration. More specifically, the predefined conditions may include the dialysate turbidity being a predefined percentage, such as 10%, above the average of the historical dialysate turbidity over the past few days, such as 3 to 5 days, a week, or longer. In another example, the predefined conditions may include the dialysate turbidity being outside of the mean of the historical dialysate turbidity over the past few days, such as 3 to 5 days, a week, or longer, by a predefined number of standard deviations, such as 1, 2, or 3 standard deviations. Upon determining that the dialysate turbidity satisfy the predefined conditions, the device 200 may trigger an alert or warning that informs the patient 102 that he/she is preliminarily indicative of peritonitis.

The device 200 provides a more objective way of measuring the turbidity of the waste dialysate fluid 130 and enables earlier and/or more accurate detection of infection such as peritonitis in the patient 102. Compared to manual visual observation by patients 102 where there is inconsistency in the quality of checking, the device 200 can reduce the risk of false positives and missing out true peritonitis cases. Moreover, the device 200 is small and can be easily integrated with the apparatus 100, compared to existing bulky and costly commercial turbidity meters that measure fluid turbidity in a static state.

On the other hand, turbidity is not a specific indicator of peritonitis and can be attributed to other factors. For example, turbid appearance of the waste dialysate fluid 130 can be caused by non-pathogenic processes such as general immune reaction, spontaneous fibrin generation, and pneumoperitoneum. A high-fat diet of the patient 102 may also result in accumulation of lipoproteins and triglycerides, inducing a milky-white coloured dialysate and confounding the visual diagnosis of peritonitis. Hence, the device 200 is useful as an objective way for early screening for peritonitis, which should be followed by another check to confirm whether the patient 102 is indeed suffering from peritonitis.

The confirmation check is typically performed using a standard peritonitis detection strip, such as a leukostix reagent strip, which can specifically respond to the presence of leukocytes esterase mainly released by neutrophils, a type of leukocytes. Peritonitis is usually associated with an increase in both the number and percentage of neutrophils in the waste dialysate fluid 130. A clinical standard of more than 100 cells/μL with more than 50% neutrophils is normally used for peritonitis detection. Additionally, neutrophils amounting to more than 50% of leukocytes in the waste dialysate fluid 130 is a strong indicator of peritonitis, even if the absolute leukocytes count is less than 100 cells/μL. Normally, the patient 102 dips the reagent strip into collected sample of the waste dialysate fluid 130. The patient 102 has to take out the reagent strip as soon as the reagent strip is wetted with the waste dialysate fluid 130, wait for a few minutes before interpreting the colour change of the reagent strip. Any significant deviation from the protocol, such as dipping the reagent strip for too long or interpreting the colour change too early or too late, would likely result in misinterpretation of the detection result.

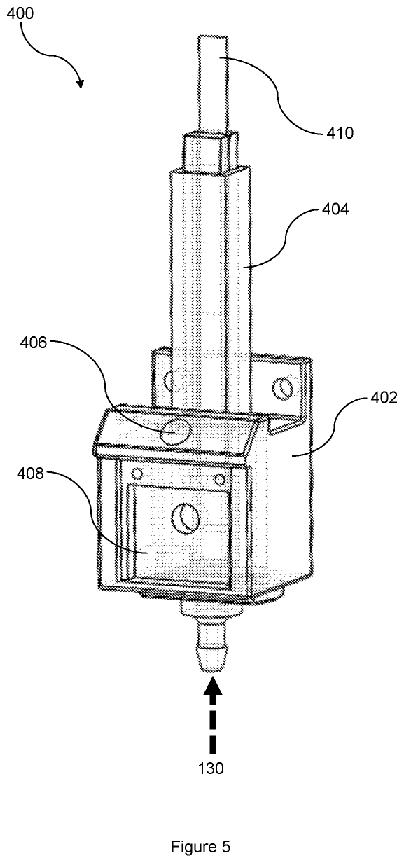

In some representative or exemplary embodiments of the present disclosure, with reference to FIG. 5, there is a device 400 for use with the peritoneal dialysis apparatus 100 for detecting infection in a patient 102 undergoing peritoneal dialysis. The device 400 includes a housing module 402 removably coupleable to a fluidic element 404 configured for receiving waste dialysate fluid 130 from the patient 102.

The fluidic element 404 may have various cross-sectional shapes such as cylindrical. The device 400 optionally includes a set of lighting elements 406 disposed on the housing module 402 and configured for emitting light into the fluidic element 404. The device 400 includes a colour sensor 408, such as an RGB colour sensor, disposed on the housing module 402. The colour sensor 408 is configured for measuring colour information of a reagent test element 410, such as a leukostix reagent strip, disposed in the fluidic element 404, wherein the reagent test element 410 has reacted with the waste dialysate fluid 130 in the fluidic element 404 which caused a colour change in the reagent test element 410. The reagent test element 410 may be illuminated by the lighting elements 406 if present and/or by ambient lighting. The device 400 includes a control module configured for detecting infection, such as peritonitis, based on the colour information, the colour information representing enzyme activity in the waste dialysate fluid 130 that is indicative of infection in the patient 102.

In some embodiments, the lighting elements 406 include one or more light emitting diodes (LEDs). For example, the LEDs may include white light LEDs. White light is generally preferred to accurately measure the colour information of the reagent test element 410.

The fluidic element 404, which includes the reagent test element 410, is a disposable component which is replaced with every use of the apparatus 100. The device 400 is reusable for the next disposable fluidic element 404 for the next peritoneal dialysis treatment. The fluidic element 404 may be pre-sterilized using a suitable method before using it for peritoneal dialysis treatment. The fluidic element 404 may be pre-installed as part of the disposable tubings used in the apparatus 100. The patient 102 needs to couple the device 400 to the fluidic element 404 before beginning peritoneal dialysis treatment. The reagent test element 410 may be manually inserted by the patient 102 into the fluidic element 404 before using the device 400. Alternatively, the reagent test element 410 may already be pre-inserted in the fluidic element 404, for example the fluidic element 404 is manufactured with the reagent test element 410 sealed inside.

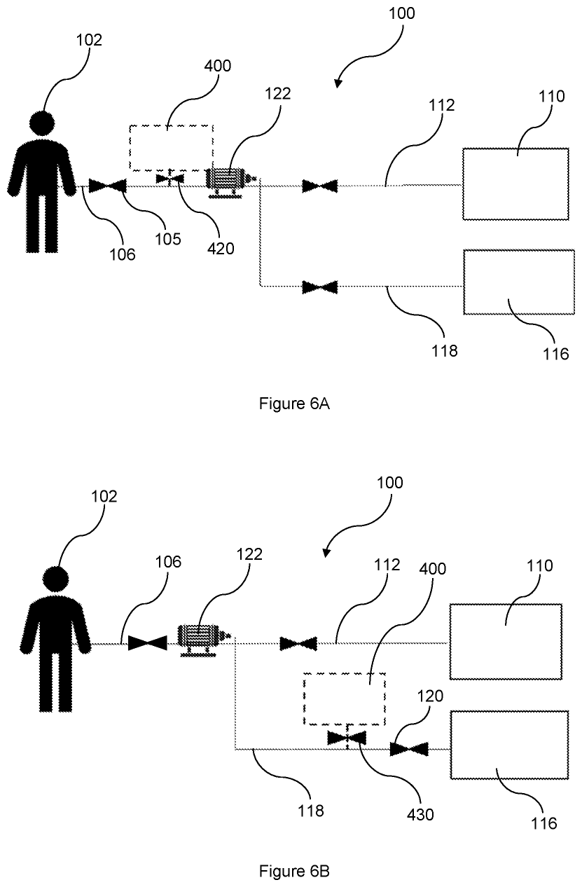

In one embodiment as shown in FIG. 6A, the fluidic element 404 is part of or connected to the common line 106 and the patient 102 couples the device 400 to the common line 106. In one embodiment as shown in FIG. 6B, the fluidic element 404 is part of or connected to the drain line 118 and the patient 102 couples the device 400 to the drain line 118.

The patient 102 can use the device 400 to measure colour information of the reagent test element 410 that has reacted with the waste dialysate fluid 130 discharged away from the patient 102 along the common line 106 and drain line 118 to the drain bag 116. In one example, the patient 102 can use the device 400 to measure the colour information during the initial drain phase before starting the peritoneal dialysis treatment. In another example, the patient 102 can use the device 400 to measure the colour information during the peritoneal dialysis treatment as the waste dialysate fluid 130 flows along the common line 106 and drain line 118 to the drain bag 116. In another example, the patient 102 can use the device 400 to measure the turbidity during the final drain phase at the end of the peritoneal dialysis treatment.

Further, to control the wetting time of the reagent test element 410, the fluidic element 404 may include a flow control mechanism configured for selectively communicating the waste dialysate fluid 130 into and out of the fluidic element 404. The device 400 may be coupled to the common line 106 or the drain line 118. The fluidic element 404 cooperates with the common line 106/drain line 118 to selectively control inflow of the waste dialysate fluid 130 from the common line 106/drain line 118 into the fluidic element 404 and outflow of the waste dialysate fluid 130 from the fluidic element 404 after a predefined duration. The predefined duration defines the wetting time of the reagent test element 410, such as ranging from 0.5 to 2 seconds, and ensures that the reagent test element 410 is properly wetted.

In one embodiment as shown in FIG. 6A, the device 400 is coupled to the common line 106. A valve 420 connects the common line 106 to the fluidic element 404 to control the flow of the waste dialysate fluid 130. The device 400 is hermetically sealed so when the patient valve 105 closes, the valve 420 opens, and fluid flow direction is reversed, causing pressure to build up in the device 400. This pressure compresses the air inside the device 400 and causes the dialysate level to rise, thus wetting the reagent test element 410. The valve 420 then closes to ensure that the reagent test element 410 is properly wetted after the predefined duration. Afterwards, the patient valve 105 remains closed, flow is reversed, and the valve 420 is opened. This releases the pressure in the device 400 through the common line 106 and retracts the waste dialysate fluid 130 away from the device 400.

In one embodiment as shown in FIG. 6B, the device 400 is coupled to the drain line 118. A valve 430 connects the drain line 118 to the fluidic element 404 to control the flow of the waste dialysate fluid 130. The device 400 is hermetically sealed so when the drain valve 120 closes, the valve 430 opens, and pressure is build up in the device 400. This pressure compresses the air inside the device 400 and causes the dialysate level to rise, thus wetting the reagent test element 410. The valve 430 then closes to ensure that the reagent test element 410 is properly wetted after the predefined duration. Afterwards, the drain valve 120 opens followed by the valve 430. This releases the pressure in the device 400 through the drain line 120 and retracts the waste dialysate fluid 130 away from the device 400.

The control module, which includes a computer processor, is configured for performing a computerized method for detecting infection such as peritonitis based on the colour information of the reagent test element 410. The method includes a step of controlling the colour sensor 408 to measure the colour information of the reagent test element 410 that has reacted with the waste dialysate fluid 130 over the predefined duration. The method includes a step of measuring the colour information of the reagent test element 410 for a predefined period after the predefined duration. The predefined period, which may range from 0.5 to 5 minutes after the predefined duration, ensures that the reagent test element 410 is measured within the right time window. The method includes steps of extracting RGB colour data from the colour information, detecting infection in the patient 102 based on the RGB colour data, wherein the RGB colour data represents enzyme activity that is indicative of infection in the patient 102.

In some embodiments, the method includes a step of converting the extracted RGB colour data into second colour data in a second colour space and determining the enzyme activity based on second colour data, wherein the second colour data represents the enzyme activity. The second colour space may include CIELAB, CIE XYZ, or YCbCr colour space.

In one embodiment, the RGB colour data is converted into the CIELAB colour space. The CIELAB colour space, also referred to as L*a*b* colour space, is defined by the International Commission on Illumination (CIE) and expresses colour as three values—L* for light intensity (black to white), a* for colour spanning from green to red, and b* for colour spanning from blue to yellow. The a* and b* values represent the four unique colours of human vision—red, green, blue, and yellow. The CIELAB colour space is thus closer to the visual sensation nature of the human eye.

The method may include denoising the RGB colour data before converting the denoised RGB colour data into the CIELAB colour space. For example, the RGB colour data may be denoised using methods such as filtering and/or outlier removals. For example, outlier removals may include removing RGB colour data beyond the third interquartile range. The method may include deriving an index parameter from the CIELAB colour data converted from the denoised RGB colour data and determining the enzyme activity based on the index parameter. More specifically, the RGB colour data from within the predefined period is converted into a plurality of samples of CIELAB colour data. For each sample, the mean of the a* values is removed and the absolute value of a* (with the mean removed) is calculated. The absolute values of a* of the samples for the predefined period are summed and then normalized by the number of samples. The index parameter is derived from the normalized absolute values of a*.

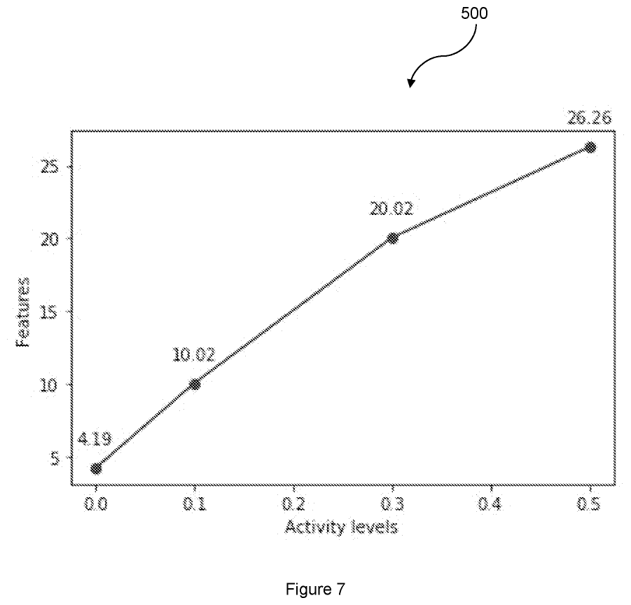

As shown in the graph 500 in FIG. 7, the index parameter is correlated with the enzyme activity. The enzyme activity may relate to activity levels of leukocyte esterase in dialysate. Leukocyte esterase activity is correlated with neutrophil amount and serves as a surrogate marker which indicates the neutrophil amount present in the dialysate. Hence, enzyme activity in the waste dialysate fluid 130 can be determined based on the determined index parameter and the graph 500, wherein the enzyme activity can be indicative of peritonitis.

An advantage of using the CIELAB colour space is that it is almost independent of the colour intensity (resulting from the light intensity from the lighting elements 406) and is more dependent on colour differentiation. The graph 500 shows that different leukocytes esterase enzyme activity levels can be well differentiated based on the index parameter. This provides for higher resolution of differentiating leukocytes esterase activity by the index parameter and reduces subjectiveness of the measurement, compared to the conventional way of using a few colour palettes as reference which can be difficult for patients 102 to differentiate those colours. A threshold may be empirically developed from clinical data to diagnose positive and negative peritonitis cases. The threshold of positive case diagnosis can be fine-tuned to balance between sensitivity and specificity of peritonitis detection.

In one embodiment, the RGB colour data is converted into the CIE XYZ colour space. In the CIE XYZ colour space, X represents the mixture of non-negative RGB colours, Y represents luminance, and Z represents blue channel information. A corresponding index parameter can be derived from the second colour data in the CIE XYZ colour space similarly to the index parameter from the CIELAB colour data, wherein there is minimal dependency on the luminance and removal of blue channel information.

In one embodiment, the RGB colour data is converted into the YCbCr colour space. In the YCbCr colour space, Y represents the luminance or light intensity component, Cb represents the blue-difference chroma component, and Cr represents the red-difference chroma component. A corresponding index parameter can be derived from the second colour data in the YCbCr colour space similarly to the index parameter from the CIELAB colour data, wherein the Cr component in the YCbCr colour space can be used to replace the a* value in the CIELAB colour space.

In one embodiment, the RGB colour data can be directly used to derive a corresponding index parameter to determine the enzyme activity.

In some embodiments, the devices 200,400 are used in combination in the apparatus 100 to improve the overall specificity of the infection diagnosis accuracy. The device 200 enables for early screening of infection based on turbidity of the waste dialysate fluid 130, and if the early screening is preliminary indicative of infection, this triggers the device 400 to perform a more sensitive detection of the infection. The devices 200,400 can be used in sequence so that when the turbidity is preliminary indicative of infection, the patient 102 then inserts the reagent test element 410 into the fluidic element 404 for the confirmation check. Alternatively, the patient 102 can used a fluidic element 404 that already contains the reagent test element 410. This saves the number of reagent test elements 410 used during peritoneal dialysis since the device 400 is only used when the device 200 triggers an early screening alert.

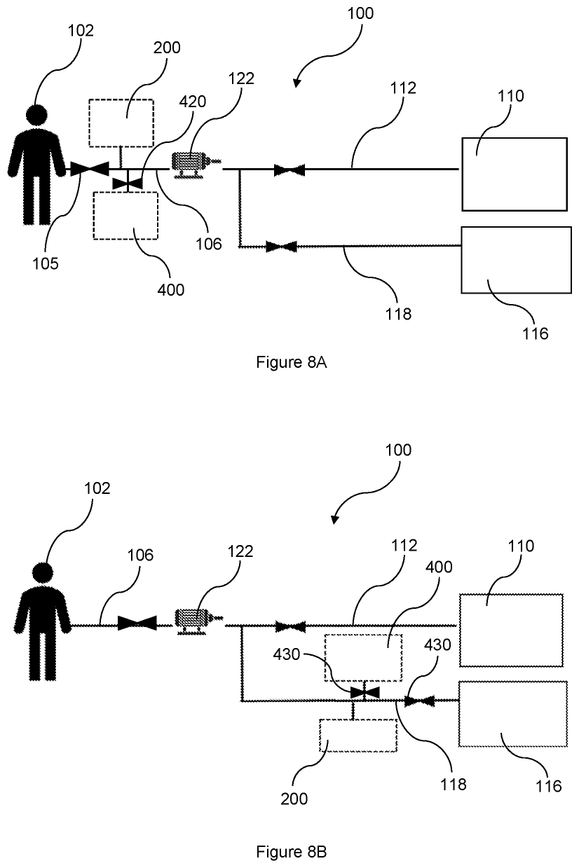

In one embodiment as shown in FIG. 8A, the patient 102 couples the devices 200,400 to the common line 106. In one embodiment as shown in FIG. 8B, the patient 102 couples the devices 200,400 to the drain line 118. The patient 102 can use the devices 200,400 during the initial drain phase before starting the peritoneal dialysis treatment, during the peritoneal dialysis treatment, or during the final drain phase at the end of the peritoneal dialysis treatment.

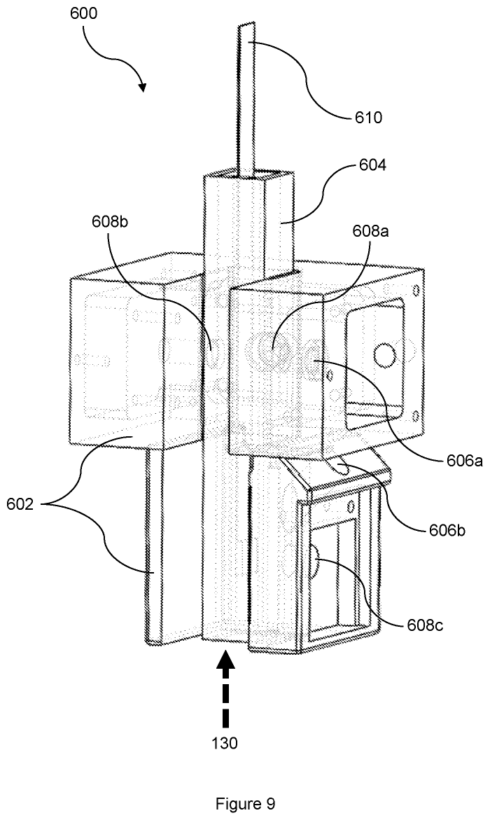

In some representative or exemplary embodiments of the present disclosure, with reference to FIG. 9, there is a device 600 for use with the peritoneal dialysis apparatus 100 for detecting infection in a patient 102 undergoing peritoneal dialysis. The device 600 is an integrated device that combines the functions of the devices 200,400. The device 600 includes a housing module 602 removably coupleable to a fluidic element 604 configured for receiving waste dialysate fluid 130 from the patient 102. The fluidic element 604 may have various cross-sectional shapes such as cylindrical. The device 600 includes a set of lighting elements 606 disposed on the housing module 602 and configured for emitting light into the fluidic element 604.

The device 600 includes a set of optical sensors 608 disposed on the housing module 602 and configured for measuring optical properties of the light that has interacted with the waste dialysate fluid 130 in the fluidic element 204 and for measuring colour information of a reagent test element 610 disposed in the fluidic element 604, wherein the reagent test element 610 has reacted with the waste dialysate fluid 130 in the fluidic element 604 which caused a colour change in the reagent test element 610. The device 600 includes a control module configured for measuring turbidity of the waste dialysate fluid 130 based on the optical properties, wherein the dialysate turbidity is indicative of infection in the patient 102 if the dialysate turbidity and historical dialysate turbidity of the patient 102 satisfy a set of predefined conditions. The control module is further configured for detecting the infection based on the colour information, the colour information representing enzyme activity in the waste dialysate fluid 130 that is indicative of the infection.

The optical sensors 608 include a scattered light sensor 608a for measuring the light that has been scattered by the waste dialysate fluid 130, and a transmitted light sensor 608b for measuring the light that has been transmitted through waste dialysate fluid 130. The optical sensors 608 further include a colour sensor 608c for measuring colour information of the reagent test element 610. It will be appreciated that the scattered light sensor 608a, transmitted light sensor 608b, and colour sensor 608c are similar to the scattered light sensor 208a, transmitted light sensor 208b, and colour sensor 408, respectively.

The lighting elements 606 include at least one first lighting element 606a cooperative with the scattered light sensor 608a and transmitted light sensor 608b. The lighting elements 606 include at least one second lighting element 606b cooperative with the colour sensor 608c. It will be appreciated that the first lighting element 606a and second lighting element 606b are similar to the lighting elements 206 and 406, respectively. Alternatively, the lighting elements 606 may include a single lighting element cooperative with all the optical sensors 608.

The fluidic element 604 may include a flow control mechanism configured for selectively communicating the waste dialysate fluid 130 into and out of the fluidic element 604 after a predefined duration. Similar to the fluidic element 404, the predefined duration defines the wetting time of the reagent test element 610, such as ranging from 0.5 to 2 seconds. When the waste dialysate fluid 130 is communicating in the fluidic element 604, the scattered light sensor 608a and transmitted light sensor 608b can measure the turbidity of the waste dialysate fluid 130. Once the waste dialysate fluid 130 is discharged from the fluidic element 604, the colour sensor 608c proceeds to measure the colour information of the reagent test element 610 that has reacted with the waste dialysate fluid 130.

The fluidic element 604, which includes the reagent test element 610, is a disposable component which is replaced with every use of the apparatus 100. The device 600 is reusable for the next disposable fluidic element 604 for the next peritoneal dialysis treatment. The fluidic element 604 may be pre-sterilized using a suitable method before using it for peritoneal dialysis treatment. The fluidic element 604 may be pre-installed as part of the disposable tubings used in the apparatus 100. The patient 102 needs to couple the device 600 to the fluidic element 604, and optionally calibrate the device 600, before beginning peritoneal dialysis treatment. The reagent test element 610 may be manually inserted by the patient 102 into the fluidic element 604 before using the device 600. Alternatively, the reagent test element 610 may already be pre-inserted in the fluidic element 604, for example the fluidic element 604 is manufactured with the reagent test element 610 sealed inside.

The patient 102 first uses the device 600 to measure colour information of the reagent test element 610 that has reacted with the waste dialysate fluid 130 during the initial drain phase which removes the waste dialysate fluid 130 from the previous dwell session. The flow control mechanism controls the inflow and outflow of the waste dialysate fluid 130 in the fluidic element 604 for the predefined duration. The patient 102 then begins the peritoneal dialysis treatment and uses the device 600 to measure turbidity of the waste dialysate fluid 130 discharged away from the patient 102, wherein the waste dialysate fluid 130 continuously flows in the fluidic element 604. In one example, the patient 102 can use the device 600 to measure the turbidity during the peritoneal dialysis treatment as the waste dialysate fluid 130 flows along the common line 106 and drain line 118 to the drain bag 116. In another example, the patient 102 can use the device 600 to measure the turbidity during the final drain phase at the end of the peritoneal dialysis treatment. The measurement results based on turbidity of the waste dialysate fluid 130 and colour information of the reagent test element 610 can complement each other can improve the overall accuracy of infection detection. The device 600 is able to detect infection such as peritonitis with greater sensitivity and specificity.

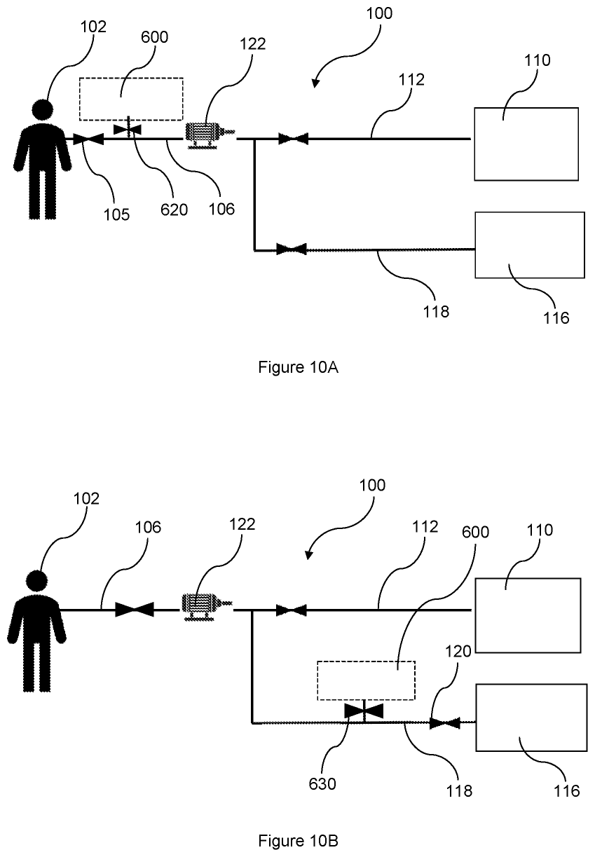

In one embodiment as shown in FIG. 10A, the fluidic element 604 is part of or connected to the common line 106 and the patient 102 couples the device 600 to the common line 106. A valve 620 may connect the common line 106 to the fluidic element 604 to control the flow of the waste dialysate fluid 130. In one embodiment as shown in FIG. 10B, the fluidic element 604 is part of or connected to the drain line 118 and the patient 102 couples the device 600 to the drain line 118. A valve 630 may connect the drain line 118 to the fluidic element 604 to control the flow of the waste dialysate fluid 130. It will be appreciated that the valves 620,630 operate similarly to the valves 420,430 described above.

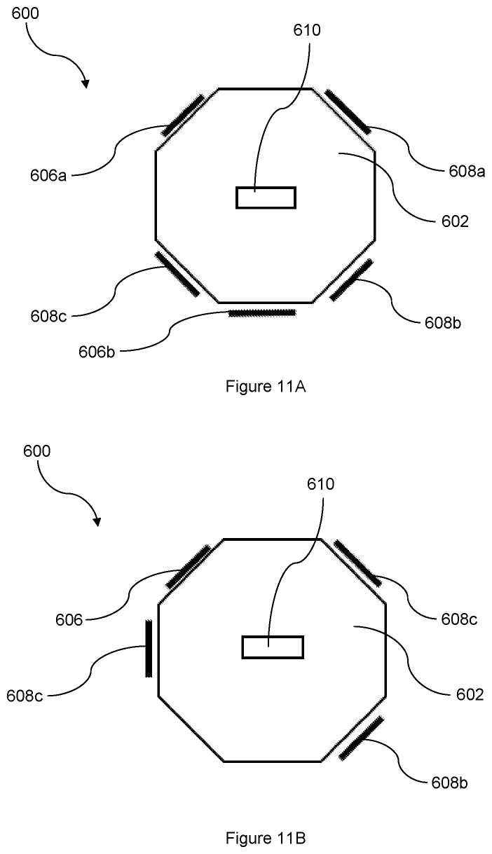

FIGS. 11A and 11B illustrate some other configurations of the device 600. Specifically, the housing module 602 may have a polygonal structure having several polygonal sides. In one embodiment as shown in FIG. 11A, the first lighting element 606a for the scattered light sensor 608a and transmitted light sensor 608b is disposed on one side and the transmitted light sensor 608b is disposed on the opposite side. The scattered light sensor 608a is disposed on another side such that it is perpendicular to the first lighting element 606a and transmitted light sensor 608b. The colour sensor 608c and second lighting element 606b for the colour sensor 608c are disposed on another two different sides. In one embodiment as shown in FIG. 11B, there is a single lighting element 606 for all the optical sensors 608. The lighting element 606 and the transmitted light sensor 608b are disposed on opposite sides of each other. The scattered light sensor 608a is disposed on another side such that it is perpendicular to the lighting element 606 and transmitted light sensor 608b. The colour sensor 608c is disposed on another side.

It will be appreciated that various aspects of the devices 200,400,600 may apply equally to each other and are not elaborated for purpose of brevity. It will also be appreciated that the control module described herein include a processor, a memory, and various other modules or components. The modules and components thereof are configured for performing various operations or steps and are configured as part of the processor. Such operations or steps are performed in response to non-transitory instructions operative or executed by the processor. The memory is used to store instructions and perhaps data which are read during program execution. The memory may be referred to in some contexts as computer-readable storage media and/or non-transitory computer-readable media. Non-transitory computer-readable media include all computer-readable media, with the sole exception being a transitory propagating signal per se.

The devices 200,400,600 thus provide improved methods of detecting infection such as peritonitis more accurately in a patient 102 undergoing peritoneal dialysis. For example, the devices 200,400 allow for early screening of preliminary indication of peritonitis which can then be confirmed with a reagent test. There is significant economic benefit to do screening then confirmation, as the screening is very cheap and costs almost nothing to the patients 102 if they use on daily basis.

Patients 102 who suffer from or are at risk of suffering from peritonitis can be identified much earlier so that medical intervention can be provided earlier. It normally takes around 4 to 10 days to treat peritonitis once diagnosed. The earlier the diagnosis, the less damage peritonitis would cause to the peritonitis membrane, and the medical treatment can be more effective and less expensive. Peritonitis is associated with higher risk of cardiovascular disease and severe infection might cause systematic sepsis which can even lead to death. Hence, early treatment means that the infected patients 102 can be more effectively treated and the mortality rate can be reduced. The devices 200,400,600 are effective in detecting peritonitis earlier and more accurately, and more patients 102 can adopt these devices 200,400,600 for home-based dialysis.

In the foregoing detailed description, embodiments of the present disclosure in relation to devices for detecting infection from peritoneal dialysis are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1-30. (canceled)

31. A device for detecting infection in a patient undergoing peritoneal dialysis, the device comprising:
a housing module removably coupleable to a fluidic element configured for receiving waste dialysate fluid from the patient;
a set of lighting elements disposed on the housing module and configured for emitting light into the fluidic element;
a set of optical sensors disposed on the housing module and configured for measuring optical properties of the light that has interacted with the waste dialysate fluid in the fluidic element; and
a control module configured for measuring turbidity of the waste dialysate fluid based on the optical properties,
wherein the measured dialysate turbidity is indicative of infection in the patient if the measured dialysate turbidity and historical dialysate turbidity of the patient, when compared to each other, satisfy a set of predefined conditions, the historical dialysate turbidity derived from dialysate turbidity measurements of the patient from a plurality of preceding days.

32. The device according to claim 31, wherein the optical sensors comprise a scattered light sensor for measuring the light that has been scattered by the waste dialysate fluid and/or a transmitted light sensor for measuring the light that has been transmitted through the waste dialysate fluid.

33. The device according to claim 32, wherein the optical properties comprise an optical ratio (y) between the scattered light and transmitted light, and wherein the device is pre-calibrated such that the optical ratio (y) and the measured dialysate turbidity (x) are substantially linearly correlated by y=mx+c, m representing a sensitivity of the device and c representing an offset value.

34. The device according to claim 31, further comprising a set of lenses for collimating the light emitting from the lighting elements into the fluidic element and/or for focusing the light on the optical sensors.

35. The device according to claim 31, further comprising the fluidic element removably coupled to the housing module.

36. The device according to claim 31, wherein the predefined conditions comprise the measured dialysate turbidity being higher than the historical dialysate turbidity averaged over the plurality of preceding days.

37. A device for detecting infection in a patient undergoing peritoneal dialysis, the device comprising:
a housing module;
a fluidic element removably coupled to the housing module, the fluidic element configured for receiving waste dialysate fluid from the patient;
a colour sensor configured for measuring colour information of a reagent test element disposed in the fluidic element that has reacted with the waste dialysate fluid in the fluidic element; and
a control module configured for detecting infection based on the colour information, the colour information representing enzyme activity in the waste dialysate fluid that is indicative of infection in the patient,
wherein the fluidic element comprises a flow control mechanism configured to selectively control inflow of the waste dialysate fluid into the fluidic element;
wherein the flow control mechanism is further configured to selectively control outflow of the waste dialysate fluid from the fluidic element after a predefined duration from said inflow of the waste dialysate fluid into the fluidic element, thereby retracting the waste dialysate fluid from the fluidic element; and
wherein the predefined duration controls a wetting time of the reaction between the reagent test element and the waste dialysate fluid.

38. The device according to claim 37, further comprising a set of lighting elements disposed on the housing module and configured for emitting light into the fluidic element.

39. The device according to claim 37, wherein the colour sensor comprises an RGB colour sensor and the control module is configured for:
extracting RGB colour data from the colour information;
converting the RGB colour data into second colour data in a second colour space; and
determining the enzyme activity based on the second colour data.

40. The device according to claim 39, wherein the control module is configured for denoising the RGB colour data and converting the denoised RGB colour data into the second colour data.

41. The device according to claim 39, wherein the control module is configured for deriving an index parameter from the second colour data, wherein the enzyme activity is determined based on the index parameter.

42. The device according to claim 39, wherein the second colour space comprises CIELAB, CIE XYZ, or YCbCr colour space.

43. A device for detecting infection in a patient undergoing peritoneal dialysis, the device comprising:
a housing module;
a fluidic element removably coupled to the housing module, the fluidic element configured for receiving waste dialysate fluid from the patient;
a set of lighting elements disposed on the housing module and configured for emitting light into the fluidic element;
a set of optical sensors disposed on the housing module and configured for:
measuring optical properties of the light that has interacted with the waste dialysate fluid in the fluidic element; and
measuring colour information of a reagent test element disposed in the fluidic element that has reacted with the waste dialysate fluid in the fluidic element,
wherein the fluidic element comprises a flow control mechanism configured to selectively control inflow of the waste dialysate fluid into the fluidic element;
wherein the flow control mechanism is further configured to selectively control outflow of the waste dialysate fluid from the fluidic element after a predefined duration from said inflow of the waste dialysate fluid into the fluidic element, thereby retracting the waste dialysate fluid from the fluidic element; and
wherein the predefined duration controls a wetting time of the reaction between the reagent test element and the waste dialysate fluid; and
a control module configured for:
measuring turbidity of the waste dialysate fluid based on the optical properties, the measured dialysate turbidity indicative of infection in the patient if the measured dialysate turbidity and historical dialysate turbidity of the patient satisfy a set of predefined conditions; and
detecting the infection based on the colour information, the colour information representing enzyme activity in the waste dialysate fluid that is indicative of the infection.

44. The device according to claim 43, wherein the optical sensors comprise:
a scattered light sensor for measuring the light that has been scattered by the waste dialysate fluid;
a transmitted light sensor for measuring the light that has been transmitted through the waste dialysate fluid; and
a colour sensor for measuring the colour information.

45. The device according to claim 44, wherein the optical properties comprise an optical ratio between the scattered light and transmitted light.

46. The device according to claim 43, wherein the optical sensors comprise an RGB colour sensor for measuring the colour information and the control module is configured for:
extracting RGB colour data from the colour information;
converting the RGB colour data into second colour data in a second colour space; and
determining the enzyme activity based on the second colour data.

47. The device according to claim 46, wherein the control module is configured for denoising the RGB colour data and converting the denoised RGB colour data into the second colour data.

48. The device according to claim 46, wherein the control module is configured for deriving an index parameter from the second colour data, wherein the enzyme activity is determined based on the index parameter.

49. The device according to claim 46, wherein the second colour space comprises CIELAB, CIE XYZ, or YCbCr colour space.

50. A computerized method for detecting infection in a patient undergoing peritoneal dialysis, the method comprising:
measuring colour information of a reagent test element that has reacted with waste dialysate fluid from the patient over a predefined duration and for a predefined period after the predefined duration;
extracting RGB colour data from the colour information from within the predefined period;
converting the RGB colour data from within the predefined period into a plurality of samples of CIELAB colour data;
deriving an index parameter from the plurality of samples of CIELAB colour data; and
detecting infection based on the index parameter, the index parameter representing enzyme activity in the waste dialysate fluid that is indicative of infection in the patient.

51. The method according to claim 50, further comprising denoising the RGB colour data and converting the denoised RGB colour data into the CIELAB colour data.
